(19) United States
(12) Patent Application Publication
(10) Pub. No.: US 2010/0100125 A1
(43) Pub. Date: Apr. 22, 2010

(54) SUTURE NEEDLE AND SUTURE ASSEMBLY

(76) Inventor: Chalam MAHADEVAN, Bangalore (IN)

Correspondence Address: BUCHANAN, INGERSOLL & ROONEY PC, POST OFFICE BOX 1404, ALEXANDRIA, VA, 22313-1404, US

(21) Appl. No.: 12/644,876

(22) Filed: Dec. 22, 2009

Related U.S. Application Data
(63) Continuation of application No. 10/586,739, filed on Oct. 23, 2006, now abandoned.

(30) Foreign Application Priority Data
Jan. 23, 2004 (IN) 53/CHE/2004

(51) Int. Cl. A61B 17/06

(52) U.S. Cl. 606/223

(57) ABSTRACT

The present invention provides a suture needle and suture assembly for use in surgery. The suture needle comprises a curvilinear, arched shaft tapering at both ends to form tips for impalement. A groove for housing the suture is provided on the needle approximately equally remotely located from the tips and running along its length either on the inner or the outer surfaces. The needle has a hole through the center of the groove and extending to the inner and outer surfaces of the shaft. A fastening means such as a crimp or plug secures the suture needle. The suture comprises a regular portion, and a narrower section housed within the groove. The hole comprises two coaxially aligned hollow cylindrical cavities with differing diameters. Fastening means is of diameter equal to the diameter of the cylindrical cavity with larger diameter so as to enable resilient fastening.

This application is a continuation of U.S. Ser. No. 10/586,739. filed Jul. 21, 2006. which is the U.S. National Stage of PCT/IN05/00027, filed Jan. 20. 2005.

### FIELD OF THE INVENTION

The invention relates to a surgical suture needle and a suture adapted therefor.

### DESCRIPTION OF THE PRIOR ART

Suture needles are used for a variety of purposes in surgery. Use can range from simple closure of surface wounds to complex anastomoses and repair of tissue and blood vessels. From time immemorial surgeons have used needles that are pointed at one end with the opposite end being used to attach the suture. The pointed end is used to impale the tissue. A portion of the suture remains in the body while the remainder of the suture is cut and the needle is discarded. Sutures are manufactured in various lengths and diameters depending on end use. Typically only about 60% to 70% of the suture provided remains in the body with the rest of the suture being discarded at the end of the procedure along with the needle or needles as the case may he.

In its most basic form, sutures are attached to needles through an eye at the end of the needle. This results in an undesirable and relatively large impalement diameter by the suture and the needle. A radical advance was the development of crimping and swaging technologies currently widely in use.

In the crimping and swaging techniques, the suture is seamlessly attached to the needle by means of crimping or swaging. By seamlessly attaching the suture to the needle, the impalement diameter becomes a function of the diameter of the needle.

Needle and suture technology has since focused on the development of needles of finer and finer diameter. To enhance visibility of needles of ever-smaller diameters introduction of alternative colors to the needle was necessitated.

Currently surgical needles are manufactured from stainless steel alloys that provide excellent ductility and high resistance to needle breaking. The pointed end of the surgical needles in current use is fashioned into various types (cutting, taper cut, etc.). These are standard designations in wide use. The development of such tips enables easier and smoother tissue penetration.

The shape of the surgical needle is of importance and a wide variety of shapes are employed. Typically, shapes range from straight to curved. Curved needles are the most commonly used, especially for surgical procedures involving delicate or fine tissue. These curved needles comprise of a shaft portion and a proximal end. The proximal end is machined to receive the suture. The other end of the shaft portion, the distal end is generally sharpened to a tip for puncturing tissue. Needles are manufactured in varying diameters. A suture appropriate for needle has a diameter that is less than the diameter of the needle.

To insert a needle manufactured in accordance with the state of the art described hereinabove into tissue, the surgeon grasps the needle at a convenient portion on the shaft using a needle holder. The tip of the needle may then be inserted into the tissue. The curvature of the needle enables the surgeon to establish a degree of control over the suturing process. After insertion of the needle into the tissue, the surgeon grasps the portion of the needle that has passed through the tissue and draws the needle and suture through the tissue. Once the surgeon is satisfied with the lie of the suture placed, the needle is grasped again using forceps or the surgeon's fingers to repoint the needle in the appropriate direction and thereafter the process continues. When a process such as this is used to approximate two hollow cavities the suture goes over the cut edge in an over and over manner. However when a hollow tube such as a vein graft requires to be attached to a fixed structure such as a coronary artery or a similar blood vessel, the initial process often involves taking separate bites in the vein followed by the artery with the vein being suspended by an assistant. In this process the needle after penetrating the vein points in the direction opposite the line needed to enter the artery. This requires the surgeon to regrasp the needle and perform a complex rotatory movement to repoint the needle in the direction needed to enter the blood vessel to be anastomosed.

While anastomosing cut ends of small intestine the surgeon often uses a method of suturing called the Connel Stitch. This suture technique involves the following steps
penetration of the suture needle at the margin of one end of the cut surface of the intestine,
entry of the needle and suture into the adjacent contiguous portion of the cut segment of intestine,
regrasping of the emerging needle and its rotation so that its tip points in the opposite direction from whence it emerged, and
reinsertion of needle into the cut portion of the intestine from the whence the needle emerged.

Regrasping and rotating movement of the needle in accordance with the state of the art is a disadvantage that the present invention seeks to overcome. In addition, features of the invention also permit for application thereof to mechanical processes.

Typically a surgical needle is made from a cut blank or length of wire of a material such as stainless steel. The blank is then shaped using well-known machining techniques to form the needle.

The present invention is directed towards a curvilinear surgical needle. The needle incorporates sharp points at both ends of the curve of the needle, with the suture being attached to the center of the needle and trailing along the leading edge in a groove (recess) cut along the length of the shaft of the needle.

Needles with tips at both ends thereof, also described as shuttle needles, are known in the art. Suture is affixed by crimping or swaging techniques to such needles through an aperture substantially equidistant from the tip ends. The aperture is provided on the inferior or lateral surface of the needle. An inhibiting factor in the use of such needles is on account of the impalement diameter in tissue, which increases significantly due to the suture protruding from the surface and its bend radius. The impalement diameter therefore is a function of the diameter of the needle, and the diameter and bend radius of the suture.

U.S. Pat. No. 5,865,836 presents a solution by providing a needle-suture combination wherein a double tipped needle is provided with an aperture disposed in between the said tips and placed laterally to receive a suture. The suture comprises of two portions, the first portion is attached to the needle and is characterized as being more pliable than the second portion. Need for pliability of the first portion is necessitated by the lateral placement of the aperture that receives the suture. Pliability is achieved by the use of multifilament or monofilament suture. The multifilament may be optimally the diameter of the second or regular portion. The monofilament first portion is narrower by about 20% to about 80% of the second portion. Further, the multifilament first portion itself is attached to the second portion by a shrinkable tubing. The invention reduces the impalement diameter by reduction in bend radius caused by modification in the suture alone. However, the suture still protrudes out from the needle and presents a drag and a sudden obstruction during suturing process, which may cause an undesirable jerk. It also results in a larger impalement diameter.

The present invention overcomes the drawbacks of prior art by reducing the shuttle needle suture diameter profile and consequently the impalement diameter in tissue so that, as with the needle having a single tip, the impalement diameter is a function of the diameter of the needle alone and not of the diameter of the needle and the suture, and the bend radius. The embodiment also provides for a smooth passage of the needle and the suture through tissue without causing undesirable drag and jerk.

### SUMMARY OF THE INVENTION

In accordance with the invention there is provided a suture needle and suture assembly for use in surgery. The suture needle comprises of a curvilinear, substantially arched shaft tapering at both ends to form tips for impalement. A groove for substantially housing the suture is provided on the needle approximately equally remotely located from the tips and running along its length either on the inner or the outer surfaces as defined herein. The needle has a hole substantially through the center of the groove and extending to the inner and outer surfaces of the shaft. A fastening means in the form of a crimp or plug secures the suture to the suture needle.

In a preferred embodiment, the suture comprises of a regular portion having a diameter appropriate to the diameter of the needle and a narrower section, which may be integrally formed or separately provided. When separately formed, the fastening means is provided on one end of the narrow section and at the other end it is attached to the regular suture portion. In either case, the narrow section is housed within the groove and has a length of at least one half of the length of the groove.

The hole through which the suture is secured comprises of two coaxially aligned hollow cylindrical cavities with differing diameters. The cylindrical cavity with smaller diameter terminates at the surface of the groove and is equal to or greater than the diameter of the suture to enable threading.

In accordance with the invention, the form of the crimp or plug remains in the cylindrical cavity with larger diameter so as to provide only the profile of the needle surface for impalement of tissue. Fastening means is of diameter substantially equal to the diameter of the cylindrical cavity with larger diameter so as to enable resilient fastening. The groove at its ends slowly decreases in depth so as to terminate at the tapering ends of the needle. The sum of the width of the suture and tapering ends of the grooves at any point is lesser than or equal to the diameter of the needle shaft at its widest. Preferably, the suture needle is a cylindrical shaft having a uniform diameter.

In a preferred embodiment, the diameter of the groove is lesser than, more preferably approximately one-third, the diameter of the shaft of the suture needle at its widest.

In yet another preferred embodiment, the suture needle is composed of titanium.

The groove recess may commence at such distance from the tip of the suture needle where the diameter of the tapering end is equal to the diameter of narrow suture. It may deepen gradually at a gradient so that the thickness of the solid portion of the needle is equal to or greater than its thickness at the point of commencement of the groove.

### BRIEF DESCRIPTION OF THE DRAWINGS

FIG. 1 is an embodiment of the surgical needle with a groove on the outer side.

FIG. 2 is an embodiment of the hole on the surgical needle with a groove on the outer side as viewed from the inner side.

FIG. 3 is a cross section of the surgical needle through the hole.

FIG. 4 is a transverse section view of the surgical needle with a groove on the outer side.

FIG. 5 is a diagram showing the surgical needle and the narrow end of suture with a groove on the outer side of the needle.

FIG. 6 is a transverse section diagram of an embodiment of the surgical needle with a groove on the inner side.

### DETAILED DESCRIPTION OF THE INVENTION

The surgical needle, hereinafter, referred to as the needle, comprises a body having a shaft, tapering ends finishing in tips for impalement, one groove on the body, a hole through the body substantially equidistant from the tips for fixing a suture to the body, and a means for securing the suture to the needle.

The body, comprising of a shaft and tapering ends terminating in tips, is a curvilinear arc when viewed transversely. For the purposes of this description, the side of the body wherein the point or points of curvature lie is called the inner side and the other the outer side. The transverse axis itself lies in a flat plane thereby giving the needle and the tapering ends a linear alignment when viewed from either of the tapering ends.

Although currently suture needles are made from stainless steel alloys, in view of the engineering requirements for manufacture of the needle in accordance with the invention and in view that the needle and suture assembly may also be employed in microsurgical procedures the use of alternative materials particularly titanium is envisaged.

The shaft may be uniformly equal in cross section through out its length. At both ends of the shaft and integrally formed therewith are tapering ends that progressively taper into tips for impalement of tissue. The tips may be engineered to form various shapes as are necessitated by surgical requirement.

A groove is provided on the shaft that extends into the tapering ends of the body. The groove accommodates the suture in a manner so as to completely house the suture. This prevents the suture from providing a drag or friction against the tissue in the suturing process.

In order to prevent excess area being presented to the tissue to be impaled, the groove may be provided to appear along the tapering ends of the body of the needle so that at the point where the suture is not housed in the groove and along the remaining length of the body towards and terminating at the tips, the cumulative broadest width of the tapering end and the suture is less than the diameter of the needle. Preferably, the groove begins to appear on the tapering ends where the diameter thereof approximates the diameter of the suture. The walls formed in the shaft as a result of the groove formation remain a constant distance apart however the depth of the groove gradually increases to its maximum and thereafter also remains constant through the remainder of the tapering end, if any, and the shaft. The groove extends through approximately 50% to 90% of the surface of the length of the needle.

Groove may be provided on the outer or the inner surface of the body, preferably on the outer. The suture is attached to the needle at the center of the groove using a swaging technique described herein.

The groove on the needle surface is machined or otherwise provided so as to enable the suture to stay implanted in the groove. The movement of the needle during the suturing process causes the suture to remain in the groove while passing through the tissue. Additionally, assistance to the surgeon is also provided and the suture is maintained under tension so that it stays out of way and remains in the groove.

The opening of the groove at the lips on the needle surface is equal to the diameter of the suture. In an embodiment, the lips are marginally smaller than the diameter of the suture. Consequently a resistance is offered to the suture while it is being placed in the groove. Preferably, the resistance is sufficient to cause the suture to ‘click’ into the space of the groove but not sufficient to cause the suture to break or to cause an operating hindrance. Once in the groove, lips offer resistance to the suture that prevents its escape from the groove during suturing process.

The depth of the groove is larger than the diameter of the suture. In a preferred embodiment, the diameter of the groove may be approximately equal to or just larger than the diameter of the suture. In such embodiment, the groove offers a snug fit for the suture in the groove, which enables the suture to remain housed within the groove during the suturing procedure.

The suture is secured to the center of the needle through the hole preferably at right angle to the groove, by fastening means. Accordingly, at any time during the suturing process the suture occupies only one half of a groove recess. The suture is housed in the groove half that is remote from the impaling end. After impalement by one end and the passing of the needle and suture through tissue, the other end of the needle is employed for the next impalement. In order to use the other end for impalement the suture is disengaged from the first half groove recess and is arranged into the groove recess that would now trail the impaling end. During the process of suturing the surgeon works with an assistant, who keeps the suture under tension in the groove and the remaining part of the suture out of the field of activity of the surgeon. Thus the surgeon would be able to reverse direction of the suture with ease, depending on which end of the needle is being used.

The groove may typically be circular in cross section. The curvature of the circular groove forms the lips on the body of the needle. The cross section of the groove may also be elliptical. Circular and elliptical cross sections are preferred since the suture is circular. Circular or elliptical cross-section offer greater surface of the walls forming the groove for interaction with the suture and are therefore preferred.

In another embodiment, the groove may be provided forming substantially straight walls on the body of the needle that are converging towards each other so that at the surface of the needle the separation therebetween is equal to or marginally lesser than the diameter of the suture. The groove essentially serves to house the suture within the body of the needle and the cavity formed thereby at its broadest must be at least equal to or greater than the diameter of suture.

The depth of the groove is primarily a function of two factors. It must be sufficiently deep to house the suture and, the thickness of the needle shaft itself should permit the groove of desired depth without sacrificing strength required of the needle in suturing process. Where the needle shaft body is widest at the midpoint of its length and gradually tapers into tips, depth of the grooves may also be made correspondingly variable in that it is the deepest at the midpoint and gradually decreases to be zero at the two ends proximal to the tips. Hence the depth of the groove should be the least possible while preserving its relationship with the suture.

A hole is provided substantially through the middle of the groove and extends through to the inner and the outer sides of the shaft thereby providing a cylindrical hollow cavity. The hole together with a means for securing suture, typically in the form of a crimp or a plug, secures the suture to the needle. The hole may comprise of two co-axial cylindrical hollow cavities of differing diameters. The diameter of the cylindrical hollow cavity having the smaller diameter is equal to or marginally larger than the diameter of the suture to enable threading. The cylindrical cavity having the smaller diameter opens into the groove, whether the groove is on the inner side or on the outer side, while the cylindrical cavity with the larger diameter opens onto the other side. In another embodiment, the hole is a hollow cylindrical cavity of uniform diameter.

Typically, there exists a standard with regard to diameter for the regular suture and not the needle. Although this is the case, there is a relationship between the needle and the diameter of the regular suture used in accordance with the state of the existing art; however, this relationship is not defined. In general ratio between the needle diameter and the diameter of the regular suture is about 2:1.

In an embodiment, suture at the end to be fastened to the needle through the hole is provided with a diameter substantially lesser than the diameter of the regular suture, which is appropriate to the diameter of the needle at its widest. The narrowed end of the suture permits a correspondingly smaller groove diameter and depth as well as the diameter of the cylindrical cavity having a smaller diameter, which in turn preserves sufficient body material and therefore the strength of the needle. The narrow section may be provided where the suture appropriate to needle has a diameter that is more than one-third the diameter of the needle. The narrowing may be provided so that the relationship between the diameter of the narrow section and diameter of the needle is approximately 1:3.

The length of the narrower section of the suture is no less than one half of the length of the groove. This is because at any given time during the suturing process, the suture occupies only one half of the groove recess. The narrower section of the suture may be formed integrally with the main body of the regular suture, which gradually tapers into the narrow section. Typically the length of the narrow section is about one to two centimeters. The narrow end may be narrower by upto 50% of the selected suture diameter. It would be appreciated that if the circumstances so require the diameter of the narrow section may be reduced even more, or, as the case may be, increased.

Preferably, the suture is fixed and held in place by a fastening means. Such fastening means may be a crimp or a plug that are, respectively, incorporated thereon or formed integrally therewith. The form of the fastening means occupies the cylindrical cavity having the larger diameter. Hence, the width of the fastening means when within the cylindrical cavity of larger diameter is such as to provide for resilient fastening of the means with the needle. In an embodiment, a cylindrical body is provided for the fastening means so that it may utilize all the surface area offered by the walls of the cylindrical cavity with the larger diameter. In order to ensure that the securing means does not provide an obstruction to the surgical process by way of protrusion beyond the surface of the outer or inner sides, the depth thereof is equal to or less than the depth of the cylindrical cavity of wider diameter. The suture may also be secured to the needle through a hollow cylindrical cavity with uniform diameter. The diameter of the cylindrical cavity is selected in accordance with the desired suture diameter for housing in the groove. In an embodiment, the hole through which the suture is secured comprises of two coaxially aligned hollow cylindrical cavities with differing diameters. The cylindrical cavity with smaller diameter terminates at the surface of the groove and is equal to or greater than the diameter of the suture to enable threading. Preferably the ratio of depth of the hollow cylindrical cavity with smaller diameter to depth of the hollow cylindrical cavity with larger diameter is 5:3 and that of their diameters is 2:1. The suture is affixed by an adhesive or by crimping of the needle shaft, or a combination thereof.

Where the fastening means is a crimp, a cylinder with a hollow coaxially aligned with the hole, is provided to receive the suture therein. In order to fix the suture to the crimp the suture is inserted into the hollow of the crimp, which is compressed to secure the suture thereto. The diameter of the cylinder after compression provides for resilient fastening with the cylindrical cavity when housed therein. In a preferred embodiment, the hollow extends through half of the depth of the cylinder. The hollow part of the cylinder is crimped with the suture inside and the solid portion is fashioned for secure fitting with the cylindrical cavity having the larger diameter.

The plug is integrally formed at one end of the narrowed suture and has a volume equal to the volume of the cylindrical cavity with the larger diameter and is substantially of the same shape so as to enable a secure and resilient fit within the cavity. The suture is provided in two detached segments, the narrow section and the regular suture. The free end of the narrow suture is first passed through the cylindrical cavity having the larger diameter and then through one having the smaller diameter. The plug formed on the trailing end is accordingly lodged into the cylindrical cavity with the larger diameter. The regular suture is attached to the narrow section at its free end by, for example, ultra sonic welding or as disclosed in U.S. Pat. No. 5,865,836. The plug is formed of the same material as the suture, for example, non-braided expanded polypropylene. The invention envisages use of suture grade materials whether bioabsorbable or not. Typically bioabsorbable materials may be polymers of glycolide, lactide, p-dioxanone, caprolactone, trimethylene, carbonate, and physical and chemical combinations thereof. Non-bioabsorbable materials include silk, nylon, polyolefin, polyester, linen and cotton.

For secure fastening, the invention also envisages use of adhesives to be applied on the fastening means and/or on the walls of the needle forming the cylindrical cavity of larger diameter. The needle and suture assembly are provided for surgery in a pre-threaded form.

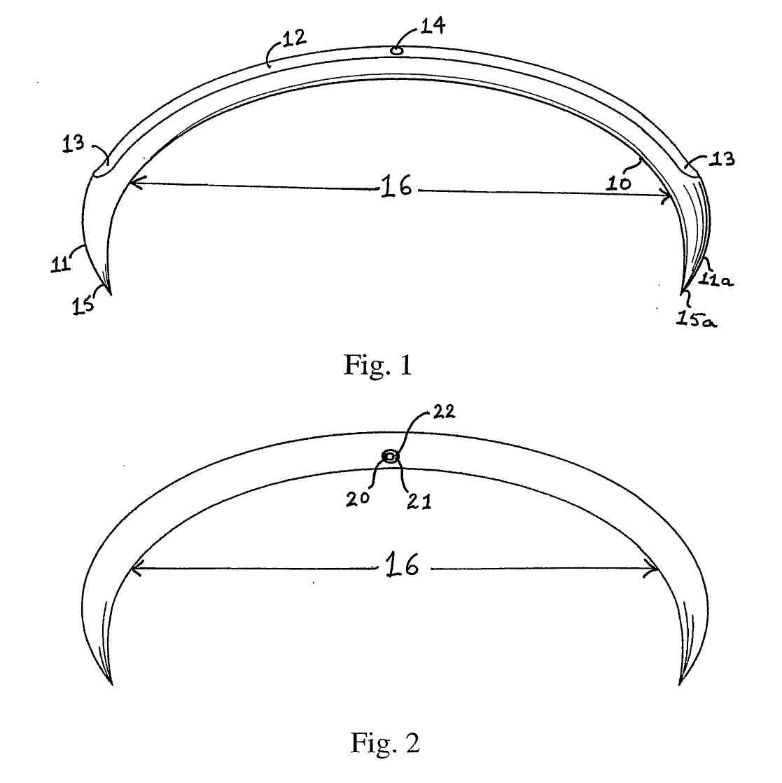

The embodiments of the invention are illustrated in drawings wherein FIG. 1 relates to an embodiment of the needle with a groove (12) provided on the outer side. The body of the needle (10) has a shaft portion (16), ends of the shaft having tapering ends (11, 11a) tapering to tips (15,15a) for impaling tissue. A groove (12) on the outer side being provided and running through the length of the shaft (16) and into the integrally formed tapering ends (11, 11a). According to one aspect of the invention, the groove (12) appears on the tapering ends (11, 11a) where the diameter of the said tapering ends is approximately twice the diameter of the suture. The groove deepens in a slope (13) to a depth sufficient to house a suture. Through the middle of the body (10) on the shaft (16) and aligned substantially so as to open on the inner and outer surfaces is provided a hole (14) through which a suture is threaded. The hole (14) on the outside interfaces with the groove (12) and at that point has a diameter sufficient to enable threading.

FIG. 2 more particularly describes the hole (14) of the embodiment shown in FIG. 1. Here the hole (14) is viewed from the inner side. The hole (14) comprises of two coaxially aligned hollow cylindrical cavities of differing diameters. The cylindrical cavity having the larger diameter opens onto the inner side of the shaft (16). Formed at the other end of the said cavity is the hollow cylindrical cavity of lesser diameter (20). Both of said cavities abut against each other so as to form a through and through cavity for suture to pass through. The diameter of the hollow cylindrical cavity of lesser diameter (20) is equal to or fractionally larger than the diameter of the suture for use with the needle. A step formation (22) on the interface of the hollow cylinders (20, 21) creates obstruction for a crimp or plug so that it may rest thereagainst.

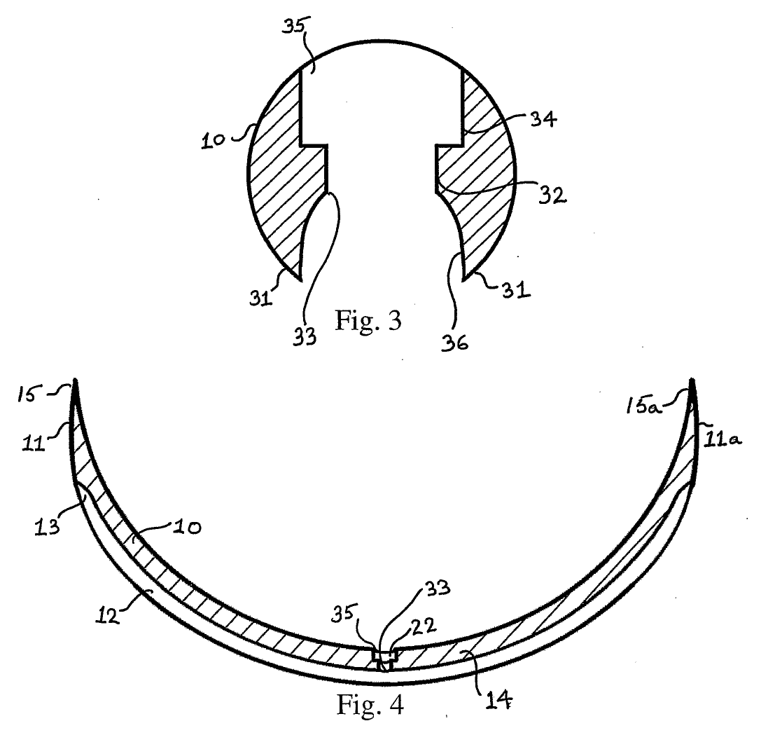

FIG. 3 is a cross section view of the hole (14) and the shaft (16) of the embodiment shown in FIG. 1. Groove (36) on the outer side of the needle body (10) is formed by the walls of the needle that finish in lips (31) on the peripheral surface of the body (10). The diameter of the groove (36) is sufficiently large so as to house a suture of desired diameter. In order to minimise the diameter of impalement to the diameter of the needle, the groove (36) is of a depth that would be greater than the diameter of the suture. The distance between the lips is equal to or fractionally smaller than the diameter of the suture. The hole (14) of FIG. 1 comprises of two coaxial cylindrical cavities of unequal diameters that are aligned so as to be substantially perpendicular to the inner and outer surfaces. The cylindrical cavity with the smaller diameter circumscribed by diameter (33) and wall (32) opens onto the deep-end surface of the groove (36). The cylindrical cavity defined by a larger diameter (35) and wall (34) opens onto the peripheral surface of the inner side. A fastening means on a suture is accommodated in the cylindrical cavity with the larger diameter (34,35).

The transverse section of the needle is shown in FIG. 4. The embodiment relates to a substantially arched needle on which a groove is machined or otherwise provided on the outer side so as to commence at the tapering ends (11,11a). The groove (12) gradually decreases in depth (13) upto the surface of the tapering ends (11, 11a). At its regular depth, that is to say that when the depth is not decreasing at the ends thereof, the groove (12) is sufficiently deep to accommodate a suture suitable for use with the needle. Tapering in this embodiment has been provided in such a manner that the tapering ends (11,11a) reduce so as to finish in tips (15,15a) that lie on the curved plane of the inner surface. The opening of the groove (12) onto the surface of the tapering ends (11, 11a) is such that the sum of diameter of the suture and of the diameter of the tapering ends at the point of emergence of the suture out of the groove or at any point after upto the tip of the needle is less than the diameter of the needle at the widest.

Hole (14) of FIG. 1 comprising of the cylindrical cavity with differing diameters is provided substantially through the center of the needle body (10) and through the shaft. The cylindrical cavity with the larger diameter (35) opens onto the inner side while the cylindrical cavity having the smaller diameter (33) opens into the groove machined on the outer side. Step (22) provides for obstruction offered to a plug or crimp on the suture to enable the suture to remain stationery on application of tug on the suture.

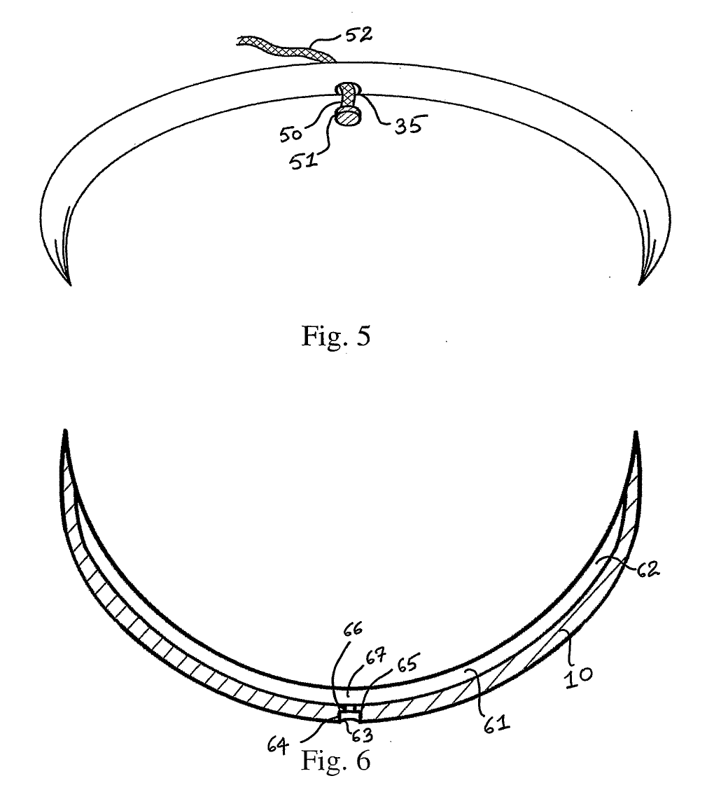

FIG. 5 is a side view of the needle and suture assembly. A groove in this embodiment is provided on the outer side of the body (not shown). The fastening means and suture assembly (50) comprises of a crimp or a plug (51) and a suture (52). The plug/crimp (51) of dimensions substantially equal to the cylindrical cavity having the larger diameter (35) is provided for resilient fastening therewith. The suture (52) is threaded through the cavities and housed in the groove.

FIG. 6 provides a transverse view of the needle with a groove (61) on the inner side. Body (10) comprising of tapering ends finishing in tips for impalement and shaft houses the groove (61). The groove (61) commences on the two tapering ends and gradually deepens to a depth larger than the diameter of the suture so that during the impaling process the suture remains housed in the groove. Substantially through the center of the needle, hole (14) of FIG. 1 is provided, that comprises of two co-axially aligned hollow cylindrical cavities of diameters in accordance with the previous embodiments. The cylindrical cavity with the smaller diameter opens into the groove. The walls forming the cylindrical cavities of differing diameters (64,66) at their interface form a step (65) that provides abutment to a crimp or plug at the end of the suture.

It is believed that the assembly of the present invention and the method of its manufacture with many of its attendant advantages will be understood after inspection of the specification and the drawings so as to enable a person of skill in the art, to whom this disclosure is addressed, to practice it without undue experimentation. While exemplary embodiments of the invention have been illustrated and described, it will be clear that the invention is not so limited and other alternatives, modifications and variations in form or construction and materials may be made without departing from the concept, spirit or scope, and without sacrificing or restricting any of its advantages. The invention may be modified within the scope defined by the appended claims.

I claim:

1. A suture needle assembly comprising a suture needle and suture:
said suture needle comprising:
(1) a curvilinear, substantially arched shaft comprising tapered ends forming two tips for impalement of tissue;
(2) an inner surface and an outer surface, wherein the side of the shaft wherein the point or points of curvature lie is called the inner side and the other side of the shaft is the outer side;
(3) a groove on either the inner or outer surface of the needle approximately equally remotely located from the said tips along the length of the needle shaft for substantially housing the suture, wherein the groove forms a cavity having a bottom and an opening between the groove and each tapered end of the shaft; and
(4) a hole from the bottom of the cavity formed by the groove and extending through the shaft to the opposite surface of the shaft for securing the suture;
wherein said suture is secured to the needle through the hole in the shaft of the needle by a means for fastening, said means for fastening comprising a crimp or plug.

2. An assembly according to claim 1, wherein said groove is on the outer surface of the needle.

3. An assembly according to claim 1, wherein said hole is located substantially at the lengthwise center of said groove.

4. An assembly according to claim 1, wherein said suture comprises a broad section having a diameter approximately the diameter of the needle and a narrow section having a diameter less than the diameter of the needle.

5. An assembly according to claim 4, wherein said narrow section of the suture is integrally formed with the suture.

6. An assembly according to claim 5, wherein the fastening means is provided on one end of said narrow section of the suture.

7. An assembly according to claim 4, wherein the cavity formed by the groove is dimensioned so as to only house the suture having a diameter approximately the diameter of the needle, or the narrow section of said suture.

8. An assembly according to claim 4, wherein the end of the narrow section of the suture is housed in the groove, the length of said narrow section being at least one half of the length of the groove.

9. An assembly according to claim 1, wherein said hole comprises two coaxially aligned hollow cylindrical cavities, wherein the diameter of one of said cylindrical cavities is larger than the diameter of the smaller diameter cylindrical cavity.

10. An assembly according to claim 9, wherein the smaller diameter cylindrical cavity terminates at the bottom of the groove, said smaller diameter being equal to or greater than the diameter of the suture to enable threading.

11. An assembly according to claim 9, wherein the crimp or plug resides entirely within the larger diameter cylindrical cavity.

12. An assembly according to claim 1, wherein the crimp or the plug is circular with a diameter substantially equal to the diameter of the larger diameter cylindrical cavity so as to enable resilient fastening.

13. An assembly according to claim 1, wherein the opening between the groove and the tapered ends of the shaft is such that the sum of the diameter of the suture running along the body of the needle in the groove and the diameter of the tapered end at the point of emergence of the suture out of the groove is less than or equal to the diameter of the shaft at its widest.

14. An assembly according to claim 1, wherein the width of the groove is less than approximately one-third the diameter of the shaft of the suture needle at its widest.

15. An assembly according to claim 1, wherein, excluding the tapering ends of the needle shaft, said shaft is cylindrical, having a uniform diameter.

16. An assembly according to claim 1, wherein the suture needle is composed of titanium.

17. An assembly according to claim 4, wherein the groove cavity begins at the distance from the tip of the suture needle at which the diameter of the tapering end is equal to the diameter of the narrow section of the suture;
said groove cavity gradually deepening at a gradient so that the thickness of the solid portion of the needle is equal to or greater than its thickness at the point of commencement of the groove.

18. An assembly according to claim 4, wherein said narrow section of the suture is separately provided, where the narrow section is attached to one end of the broad section of the suture.

19. An assembly according to claim 18, wherein the fastening means is provided at one end of said narrow section of the suture.
